(19) United States
(12) Patent Application Publication
(10) Pub. No.: US 2025/0009953 A1
(43) Pub. Date: Jan. 9, 2025

(54) BLOOD VOLUME SENSOR SYSTEM

(71) Applicant: Orrum Clinical Analytics, Inc., Plymouth, MI (US)

(72) Inventors: Hao Jiang, Plymouth, MI (US); Tanner Foley, Plymouth, MI (US); Alex Fernandez-Rajal i Sabala, Plymouth, MI (US); Makenna Fockler, Plymouth, MI (US); Zahra Alzayer, Plymouth, MI (US); Ahron Wayne, Plymouth, MI (US)

(73) Assignee: Orrum Clinical Analytics, Inc., Plymouth, MI (US)

(21) Appl. No.: 18/716,274

(22) PCT Filed: Dec. 8, 2022
(86) PCT No.: PCT/US2022/052204

§ 371 Date: Jun. 4, 2024

Related U.S. Application Data
(60) Provisional application No. 63/287,141, filed on Dec. 8, 2021.

(51) Int. Cl. A61M 1/36

(52) U.S. Cl. CPC A61M 1/3624; A61M 1/3666; A61M 2205/3306; A61M 2205/3379

(57) ABSTRACT

A blood volume sensor system including a container defining a blood volume reservoir, a volume sensor configured to monitor the blood volume reservoir and output data corresponding to a volume of blood contained in the blood volume reservoir, and a digital computing device in communication with the volume sensor and configured to receive the output data regarding the volume of blood contained in the blood volume reservoir. The volume sensor being a gravimetric sensor, an optical sensor, a contact imaging sensor, a camera sensor, or a time of flight sensor. The system additionally including an oxygenator attached to the blood volume reservoir. The system being configured to be incorporated into a surgical pack of a heart lung machine to monitor the blood volume reservoir in real time to reflect a circulating blood volume of a patient.

### RELATED APPLICATIONS

This application claims priority benefit of U.S. Provisional Application Ser. No. 63/287,141, filed on Dec. 8, 2021, the contents of which are hereby incorporated by reference.

### FIELD OF THE INVENTION

The present invention relates to medical devices, and more particularly, it relates to a blood volume sensor system for determining in real time the total circulating blood volume of an extracorporeal circuit associated with a patient, thereby providing continuous monitoring of blood volume changes and trending-type monitoring.

### BACKGROUND OF THE INVENTION

The determination of total circulating blood volume (CVB) is of substantial importance for a variety of medical situations. For example, the first and most important therapeutic goal for hemorrhagic, post operative, cardiogenic, traumatic, neurogenic for septic shock is to restore blood volume to normal levels.

A broad variety of patient conditions are associated with the abnormal blood volume levels referred to as “hypovolemia” (circulating volume too low) and “hypervolemia” (circulating volume too high). Hypovolemia occurs commonly during surgery and represents a significant cause of intestinal hypoperfusion. Hypoperfusion occurs as a response to any reduction in circulating blood volume as blood is directed away from the intestinal vascular bed in favor of vital organs. Management of circulating blood volume is essential prior to, during and following cardiopulmonary bypass procedures, inasmuch as avoiding hypovolemia improves organ perfusion and reduces morbidity and mortality. Circulating blood volume data also is important for carrying out the treatment of patients with ruptured cerebral aneurysms who often are hypovolemic. Hemorrhagic shock following traumatic injury is caused by extensive blood loss or blood loss induced trauma in the central nervous system. Failure to recognize the presence or extent of blood loss is an important factor in avoiding the loss of the patient. While hypotensive injury victims routinely receive rapid fluid resuscitation, an excessive addition of fluid into the vascular system may increase bleeding and worsen the outcome, see: Silbergleit, Schultz, et al, “A New Model of Uncontrolled Hemorrhage that Allows Correlation of Blood Pressure and Hemorrhage”, Academic Emergency Medicine, Vol. 3 No. 10, pp 917-921 (1996).

Hypovolemia is one of the principal defects contributing to cardiovascular instability and circulatory failure during septic shock. During sepsis, microcirculation often is severely impaired to exacerbate the problem of hypervolemia. Hypovolemia-induced hypotension is reported to complicate approximately 30% of all dialysis treatments. Short duration hemodialysis involving ultra filtration can cause hypovolemia unless corrective action is taken such as reducing the filtration rate or interrupting the hemodialysis process to allow for compensatory changes in the patients circulating blood volume. Acute renal failure occurs most commonly in a setting of surgery and trauma due to hypovolemia, sepsis, obstetric complications, hemolytic reaction and poisoning. A principal challenge to practitioners treating burn patients is the management of circulating blood volume in the presence of excessive plasma loss at the burn sites. Hypovolemia is a common complication of patients with burns.

However, despite the substantial importance of determining the total circulating blood volume (CBV) of a patient, making accurate CBV determinations has been an elusive undertaking. Typically, other hemodynamic parameters such as mean arterial pressure (MAP), wedge pressure (WP) or occlusion pressure, central venous pressure (CVP) and hematocrit (Hct) are used by clinicians to infer blood volume. However, such inferentially based approaches do not accurately reflect blood volume except at more extreme departures from normal levels. See in this regard: Shippey, C. R., Appel, P. L., Shoemaker, W. C., “Reliability of Clinical Monitoring to Access Blood Volume in Critically Ill Patents”, Critical Care Medicine, Vol. 12, No. 2, pp 107-112 (1984)

Conventional methods for measuring circulating blood volume depend typically upon the dilution of a dye, radioactive tracer or other analyte which, following injection is mixed into the bloodstream. Blood volume then is calculated, inter alia, from the extent of dilution and such calculation assumes that the indicator-analyte is immiscible in red blood cells.

In order to estimate total circulation blood volume (TCBV), i.e., the summation of plasma volume (PV) and red blood cell volume (RBCV), the large vessel hematocrit (LVH) also is measured so that total blood volume is obtained by the following relationships:

$$Plasma Volume Measured : T C B V = \frac{P V}{1. - L V H}$$

$$Red Blood Cell Volume Measured : T B B V = \frac{R B C V}{L V H}$$

The most accurate method for measuring total blood volume avoids the potential error of using the large vessel hematocrit value (which is not representative of the hematocrit throughout the circulatory system) by separately measuring the plasma volume and red blood cell volume. This method is known as the Summation Method. See generally: Dagher et al, “Blood Volume Measurements: A Critical Study. Prediction of Normal Values: Controlled Measurement of Sequential Changes: Choice of a Bedside Method”, Advances In Surgery 1969; 1:69-109.

As has been reported in the literature since 1941, of the various radionuclides employed, a technique utilizing ⁵¹Cr has been considered a “gold standard” for deriving circulating blood volume values. However, this approach, as well as dye-based dilution approaches are both costly and are limited to relatively infrequent measurement. As a consequence, a continuous monitoring of blood volume changes or trending-type monitoring has not been available to practitioners. A more recent approach, utilizing ¹³¹I as a radiolabel provides for the obtaining of a plurality of blood samples over 20-35 minutes following tracer injection. Tracer dilution is combined with hematocrit to calculate blood volume. See in this regard: U.S. Pat. Nos. 5,024,231 and 5,529,189. In general, this approach has been problematic in terms of cost, limitations on the number of measurements which can be made, and the inherent procedure and physiologic limitations associated with the radionuclide.

Practitioners involved in the management of more critical hemodynamic conditions, typically turn to commonly monitored and thus more immediately available parameters such as mean arterial pressure (MAP), pulmonary catheter wedge pressure (PCWP), central venous pressure (CVP), heart rate (HR) and hematocrit (HCT) to estimate or infer a value for total circulating blood volume. Studies have shown, however, that such inference-based determinations are prone to error.

During cardiac Surgery, many complex medical devices are used to monitor and maintain patient physiology and provide life support during the procedure. Additionally, disposable medical devices mimic the function of the heart and lungs, including oxygenators and centrifugal blood pumps. A cardiopulmonary bypass (CPB) incorporates an extracorporeal circuit controlled through a heart-lung machine to circulate the patient's blood to provide physiological support during a cardiac surgery or critical life support. FIG. 1 shows one simplified schematic of a CPB circuit: the deoxygenated venous blood is drained from the patient's right atrium or vena cava through a venous line and sequestered inside the venous blood reservoir and flows through an oxygenator driven by a centrifugal pump; the oxygenated blood is then injected into the patient's aorta. During a CPB procedure, a perfusionist operates the heart-lung machine to manage the physiology of the patient. For the perfusionist, the volume of the venous blood stored inside the reservoir is a crucial information for making quantitative decisions on the patient's cardiac output and blood volume management, and to monitor the patient's physiological conditions. Despite the significance of this venous blood volume, the current form of volume measurement has many limitations. All existing commercial reservoirs used in clinical CPB procedures require the perfusionists to visually read the volumetric marks on the surface of the reservoirs. Such a manual approach is generally inefficient and inconvenient, because the perfusionists must pay close attention to the marks and spend a few seconds for making one reading. Furthermore, the visual-based reading is prone to human errors and has poor consistency and poor resolution (smallest discernible volume change), usually around 100 mL resolution for a blood volume larger than 1000 mL. In addition, since there is no automatic device associated with the reservoirs to record the blood volume in real time, it is unpractical to monitor and track blood volume changes over a long period. Although most commercial reservoirs are equipped with a safety sensor to detect if the blood volume drops below a critical level, it cannot provide the reading of the actual volume.

Thus, there exists a need for the ability to recognize and measure of an Extracorporeal Circuit (ECV) as surrogate measure for patient circulating volume.

### SUMMARY OF THE INVENTION

The present invention provides a blood volume sensor system that includes a container defining a blood volume reservoir, a volume sensor configured to monitor the blood volume reservoir and output data corresponding to a volume of blood contained in the blood volume reservoir, and a digital computing device in communication with the volume sensor and configured to receive the output data regarding the volume of blood contained in the blood volume reservoir. The volume sensor is a gravimetric sensor, an optical sensor, a contact imaging sensor, a camera sensor, or a time of flight sensor. The system additionally includes an oxygenator attached to the blood volume reservoir. The system is configured to be incorporated into a surgical pack of a heart lung machine to monitors the blood volume reservoir in real time to reflect a circulating blood volume of a patient.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a schematic drawings of a simplified existing cardiopulmonary bypass circuit including a venous reservoir, a centrifugal pump, and an oxygenator;

FIG. 2 is a schematic drawing of a general blood volume sensor system according to embodiments of the present invention;

FIG. 3 is a schematic drawing of a gravimetric blood volume sensor system according to embodiments of the present invention;

FIG. 4 is a graph showing an experimental result for using a gravimetric volume sensor to monitor the changing volume inside the reservoir of the system according to embodiments of the present invention;

FIG. 5A is a schematic drawing of a contact imaging sensor based optical blood volume sensor system according to embodiments of the present invention;

FIG. 5B is a schematic drawing of details of the contact imaging sensor based optical blood volume sensor system of FIG. 5A;

FIG. 6A is a schematic drawing of a contact imaging sensor based optical blood volume sensor system according to embodiments of the present invention;

FIG. 6B is a schematic drawing of details of the contact imaging sensor based optical blood volume sensor system of FIG. 6A;

FIGS. 7A-7F are graphs showing the response of the sensor of FIG. 6A array in the contact image sensor vs. changing volumes of an opaque liquid;

FIG. 8A is a graph showing calibration of the sensor of FIG. 6A in which the sensor's reading vs. count of blocked pixels are calibrated, and in which the square marks are data points measured from the calibration procedure and the solid lines are the fitted linear equations for the corresponding segments;

FIG. 8B is a graph showing calibration of the sensor of FIG. 6A in which the sensor's resolution vs. different ranges of volumes are calibrated;

FIG. 9A is a graph showing real time results of blood volume and average flow rate tracked during an experiment testing the sensor of FIG. 6A;

FIG. 9B is a screen readout provided on a venous reservoir with the blood volume sensor of FIG. 6A;

FIG. 10A is a schematic drawing of an optical blood volume sensor system according to embodiments of the present invention;

FIG. 10B is a schematic drawing of an optical blood volume sensor system according to embodiments of the present invention;

FIG. 11 a schematic drawing of a camera based blood volume sensor system according to embodiments of the present invention;

FIG. 12 a schematic drawing of a time of flight laser based blood volume sensor system according to embodiments of the present invention;

FIG. 13 a schematic drawing of a ultrasound time of flight based blood volume sensor system according to embodiments of the present invention;

FIG. 14 is a diagram showing the operation of a gravimetric sensor according to embodiments of the present invention.

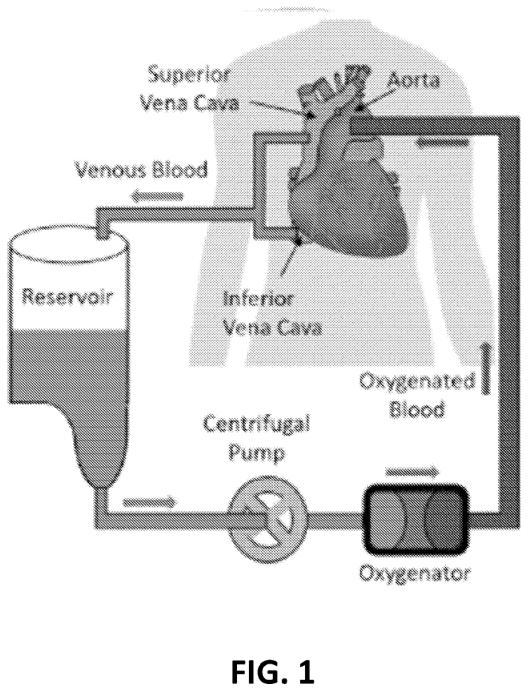

### DETAILED DESCRIPTION OF THE INVENTION

The present invention has utility as a venous blood volume sensor system for determining in real time the total circulating blood volume of an ECV as a surrogate for that of a patient. An inventive system will be integral in an automated extracorporeal pump/heart lung machine) and provide real-time data as to blood volume without reliance on error prone inference-based determinations, thereby providing continuous monitoring of blood volume changes and trending-type monitoring in real-time without reliance on error prone inference-based determinations. In a specific inventive embodiment, a measurement of dynamic blood volume in an extracorporeal circuit is made while a patient is on cardiopulmonary bypass.

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

During cardiac Surgery, many complex medical devices are used to monitor and maintain patient physiology and provide life support during the procedure. Additionally, disposable medical devices mimic the function of the heart and lungs, including oxygenators and centrifugal blood pumps. These disposable systems are incorporated into a “pack” which contains various components allowing these systems to integrate with the Heart lung machine. Perfusionists are the clinicians who are responsible for the operation of these devices during an operation.

According to embodiments, the inventive blood volume sensor system is incorporated into a surgical “pack” and thereby provides perfusionists and surgeons with the ability to recognize and measure patient circulating blood volume in real-time. Accordingly, embodiments of the inventive blood volume sensor system provide vitally important information regarding patient status that allows practitioners to quickly react and avoid “hypovolemia” (circulating volume too low) and “hypervolemia” (circulating volume too high) situations that lead to negative patient outcomes. The maintenance of the patient circulating volume status affects treatment algorithms and clinical responses which would be crucial to any clinical decision support applications. It is appreciated that an inventive system is amenable to providing data to an artificial intelligence algorithm in concert with patient diagnostics to provide a learning data set to improve operation of the bypass equipment so as to inhibit blood volumetric conditions of hypovolemia and/or hypervolemia.

According to embodiments an inventive blood volume sensor system 100 is incorporated into a disposable “pack.” The system includes a venous blood reservoir 10. According to embodiments, the venous blood reservoir 10 is configured to be attached directly to left side of a patient's heart. This arrangement allows a perfusionist to drain and sequester blood from the patient, thereby allowing a surgical team to work directly on the heart. According to embodiments, the sequestered blood is shunted from the patient and stored in the reservoir 10. Simplified, this reservoir 10 reflects patient circulating volume.

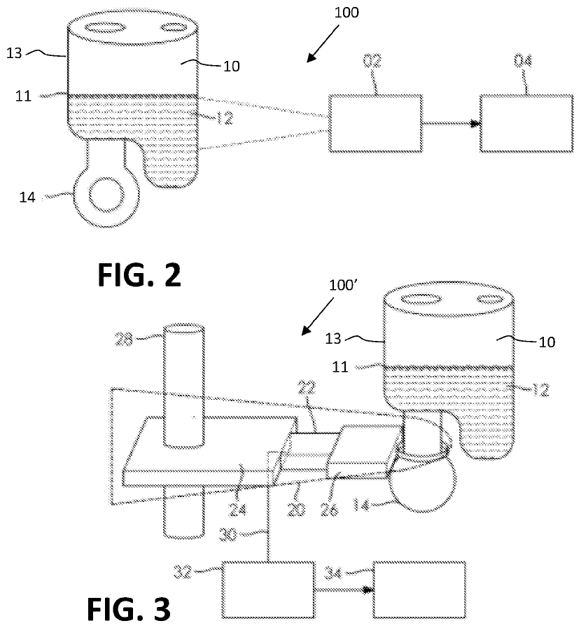

As shown in FIG. 2, the inventive blood volume sensor system 100 generally includes a container 13 defining a venous blood reservoir 10 with an attached oxygenator 14. As described above, venous blood 12 is shunted from a patient and stored in the reservoir 10. A blood/air interface 11 is defined within the reservoir 10. A volume sensor 2 monitors the reservoir 10 and measures the volume of the venous blood 12 in the reservoir 10. Such monitoring and measuring are done in real-time. The volume of the venous blood 12 as measured by the volume sensor 2 is then communicated with a computer 4 or other digital computing device for the purpose of monitoring the perfusion process. This information is then used for data analytics on patient health.

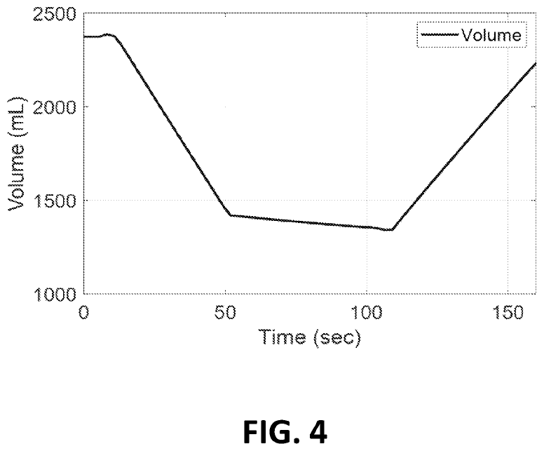

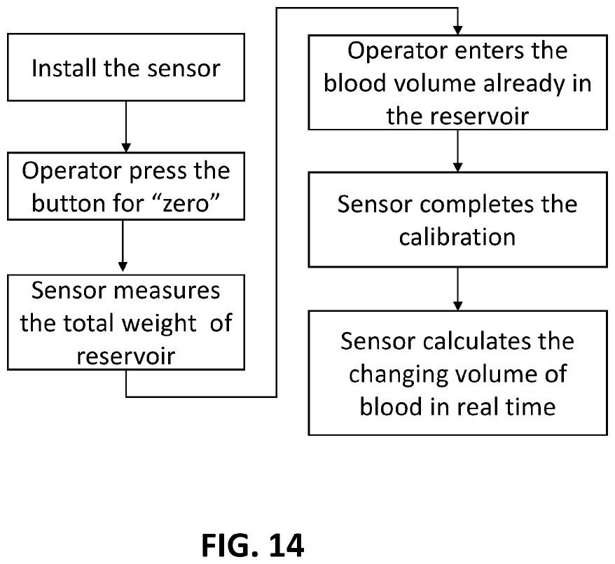

According to an embodiment as shown in FIG. 3, the volume sensor 2 of an inventive blood volume sensor system 100, as described above, is a gravimetric blood volume sensor 20. According to such gravimetric volume sensor system 100′ embodiments, the gravimetric volume sensor 20 measures the volume of venous blood 12 contained in the reservoir 10 based on the weight of the blood 12 in the reservoir 10. The operation of such a gravimetric sensor is shown in FIG. 14, which shows that once the sensor 20 is installed and zeroed, the sensor 20 measures the total weight of the reservoir 10. An operator then enters the blood volume in already in the reservoir 10, and the sensor 20 completes the calibration. The sensor 20 then calculates the changing volume of the blood within the reservoir 10 in real time. According to embodiments, the gravimetric volume sensor 20 includes a load cell 22. The load cell 22 is a mechanical sensor that measures the weight introduced by the venous blood 12 stored inside the reservoir 10. As shown in FIG. 3, the blood volume sensor system 100′ additionally includes a mounting component 26 for connecting the load cell 22 to the reservoir 10. According to embodiments, the mounting component 26 indirectly connects the load cell 22 to the reservoir 10 via connection to the oxygenator 14. According to embodiments, the gravimetric volume sensor system 100′ additionally includes a second mounting component 24 configured to connect the load cell to a support structure 28, which according to embodiments is a post or any other suitable mechanical support. Such a support structure 28 may also be the support for the entire volume sensor system 100′. The load cell 22 communicates with a microcontroller unit (MCU) 32 by way of a data cable 30 connection. The MCU 32 reads the output data from the load cell 22 and in turn communicates that data to a computer 34. According to some embodiments, the sensor system 100′ additionally includes electronic components connected to the load cell 22 to control the sensor's functions. Such electronic components may include a keypad for inputting information into the sensor system 100′, an OLED for outputting information from the sensor system 100′, and a zero button. FIG. 4 shows a graph showing an experimental result for using a gravimetric volume sensor 20 to monitor the changing volume inside the reservoir of the system 100′.

In the embodiment shown in FIG. 3, the load cell connects 22 the container 13 to the post mount 28, which according to embodiments is installed on the heart-lung machine. The oxygenator 14 is connected under the reservoir 10. The weight applied on the load cell 22 includes the container 13, the venous blood 12 inside the reservoir 10, the oxygenator 14, and tubing. From the weight measured by the load cell 22, the volume of blood is calculated. As such, before the operation, the sensor 20 must be properly “zeroed” to subtract the weight of the components other than blood. It also requires a quick calibration by entering the blood volume already inside the reservoir 10. The zero button and keypad, described above, are used to manually calibrate the sensor's 20 reading according to the current blood volume inside. The sensor's reading is displayed on the OLED display in real time, at a rate of one reading per 2.73 seconds. All electronic components are connected and controlled by microcontroller board.

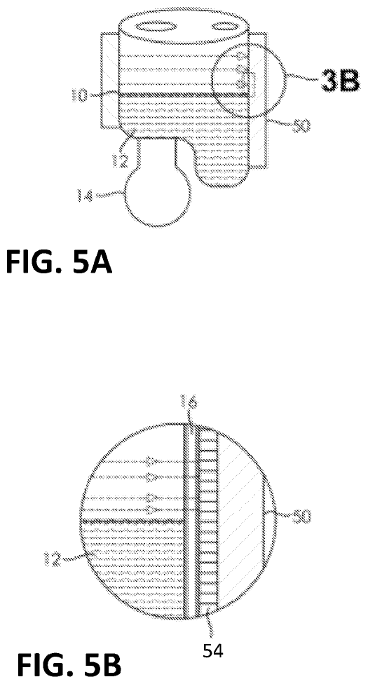

According to an embodiment as shown in FIGS. 5A and 5B, the volume sensor 2 of an inventive blood volume sensor system 100, as described above, is a contact imaging sensor (CIS) based optical sensor 50. In such CIS based optical blood volume sensor systems 100″, the CIS based optical volume sensor detects light affected by the level of blood 12 in the reservoir 10. That is, the CIS based optical blood volume sensor system 100″ includes a light source 52, such as an LED strip, and a contact imaging based optical sensor 50. According to such embodiments, the wall 16 of the container 13 that defines the reservoir 10 is transparent. The light source 52 is positioned along the reservoir 10 such that it shines light through the reservoir 10. The contact imaging based optical sensor 50 with its photodiodes (photodetectors) 54 is positioned along the reservoir 10 such that the photodiodes may detect the light emitted by the light source 52 that passes through the reservoir 10. According to some inventive embodiments, the sensor 50 includes 5000 photodiodes in a linear array. According to other inventive embodiments, the light source 52 is positioned on one side of the reservoir 10 while the contact imaging based optical sensor 50 is positioned along the opposite side of the reservoir 10. When the reservoir 10 is empty, the light 53 emitted by the light source 52 illuminates all photodiode array/photodetectors 54 in the contact imaging based optical sensor 50 on the other side of the reservoir 10. However, when a volume of blood 12 resides within the reservoir 10, the blood 12 blocks a portion of the light 53 emitted by the light source 52. That is, the incident light emitted above the volume of blood 12 is transmitted to the contact imaging based optical sensor 50 and is detected by the photodiode array/photodetectors 54 positioned above the blood 12 level. The light emitted below the volume of blood 12 is absorbed by the blood 12, and the photodetectors 54 covered by the volume of blood 12 receive very little signal. The signal from the entire array of photodetectors 54 tells the level of blood 12 in the reservoir, which based on calculations correlates to the volume of blood 12 in the reservoir 10. The contact imaging based optical sensor 50 communicates this data to a computer 4 or other suitable digital computing device, as described above.

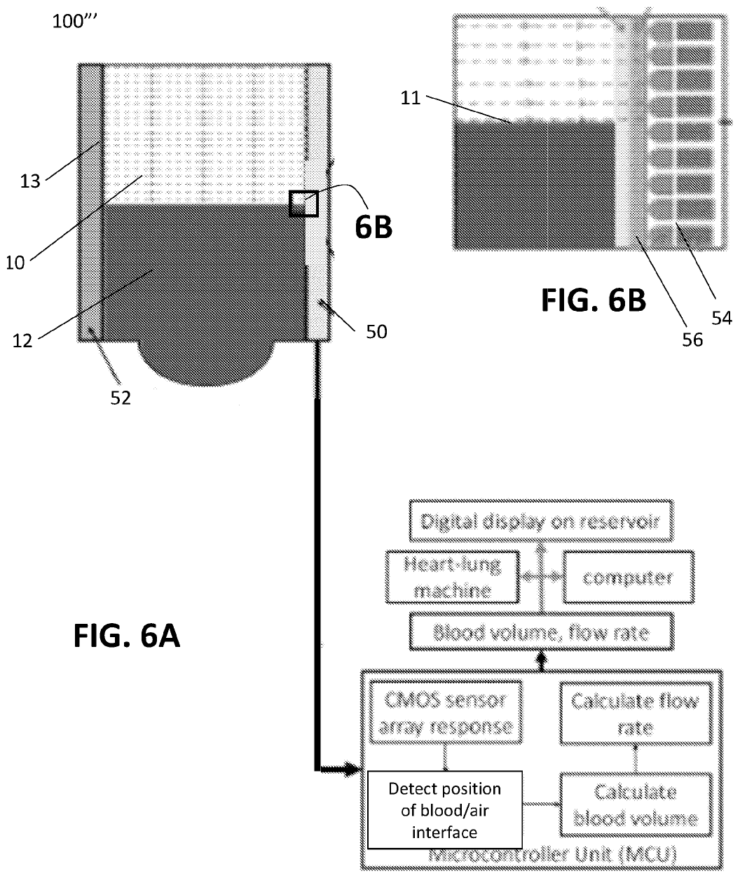

According to an inventive embodiment show in FIGS. 6A and 6B, the volume sensor 2 of an inventive blood volume sensor system 100″, as described above, is an optical scanner to automatically measure, display, and record blood volume inside the venous reservoir in real time. The core component is a high-resolution contact image sensor (CIS) based optical sensor 50. The CIS device includes a linear array of more than 5,000 CMOS sensor pixels 54 with pixel pitch about 50 μm. A CIS device 50 is an optical light-sensing system that uses light to scan objects. It is commonly used to scan documents in office scanners by detecting the light reflected from the document's surface into the CMOS sensors to form one line of image. According to inventive embodiments of the blood volume sensor, an LED light source 52 and a CIS device 50 are mounted on the two opposite sides of the reservoir with a detachable design, as shown in FIG. 6A. The light from the LED light source 52 is launched into the reservoir 10, and the transmitted light reaches the CIS device 50. Since blood is an optically opaque liquid, the transmitted light yields a stepwise pattern, which is scanned by the CIS device 50. The CMOS sensor pixels 54 below the blood level 11 are blocked by blood and give a much lower light intensity signal than those clear pixels above the blood level. To improve the optical contrast, a multilayer optical filter 56 consisted of multiple neutral density filter films and a light diffusion film is inserted before the CIS device.

The entire blood volume sensor is controlled by a microcontroller unit (MCU). By analyzing the response of the CMOS senor array, the number of blocked pixels are counted, which is linearly related on the blood level inside the reservoir. With proper calibration for the specific reservoir shape, the blood level is converted into a blood volume. With blood volumes being recorded over time, the flow rate of the net flow of blood inside the reservoir is also calculated. The results are immediately shown on a digital display mounted on the front side of the reservoir, and all data are sent to the heart-lung machine and other connected computers in real time. For each reading from the CIS device, the CMOS sensor pixels are sequentially scanned, which is controlled by a clock signal generated from the MCU. The output voltage from each CMOS sensor pixel is read by a 12-bit analog to digital conversion (ADC) on the connected MCU. The entire CMOS sensor array (comprising 5,300 CMOS sensor pixels) is scanned once in every 0.25 second, which can give 4 readings of blood volume per second. The volume readings in real time are stored in the MCU and also communicated with connected computers and the heartlung machine. The instant flow rate is calculated from two neighboring volume readings but it often contains too much fluctuations due to the noise. A more meaningful value is the average flow rate, which is calculated from volume readings sampled over a 3-second window (containing 12 readings). This average flow rate provides a stable reading to reflect the actual net flow of blood into the reservoir.

According to some inventive embodiments, the LED light source is a white LED strip (color temperature 6500 K) powered by a 24 volt DC power adapter. The intensity of light directly illuminated onto the CIS device surface was measured with a lux sensor mounted on the side of the CIS device when the reservoir was empty, and the CIS device was detached. The measured light intensity was between 3,000 lux and 5,000 lux (one lux is one lumen per square meter).

According to other inventive embodiments, the blood volume sensor relies on the optical contrast between blocked and clear CMOS sensor pixels. In the ideal scenario, the optical contrast should only come from the illumination by the LED light source. However, in practical applications, the optical contrast can be affected by the ambient light from the environment, typically in a surgical operating room. In order to minimize the interference from the ambient light, multiple layers of neutral density (ND) filter films are stacked on top of the CIS device to make a total optical density of 3.3. The ND films can effectively attenuate the ambient light into a negligible level. Since the LED light source is sufficiently strong, it can still trigger a high amplitude signal in the CMOS sensor pixel after transmitting through the ND films.

According to still other inventive embodiments, the blood volume sensor implements a CIS device to optically scan the blood inside the reservoir to find the liquid level of the blood, which is then converted into the volume. This conversion from liquid level into volume is crucial for accurate reading and it depends on the actual shapes of the specific reservoirs being used upon. For each type of reservoir, a calibration must first be done for once and the calibration coefficients will be stored in the MCU and applied for the same type of reservoir when measuring the blood volume.

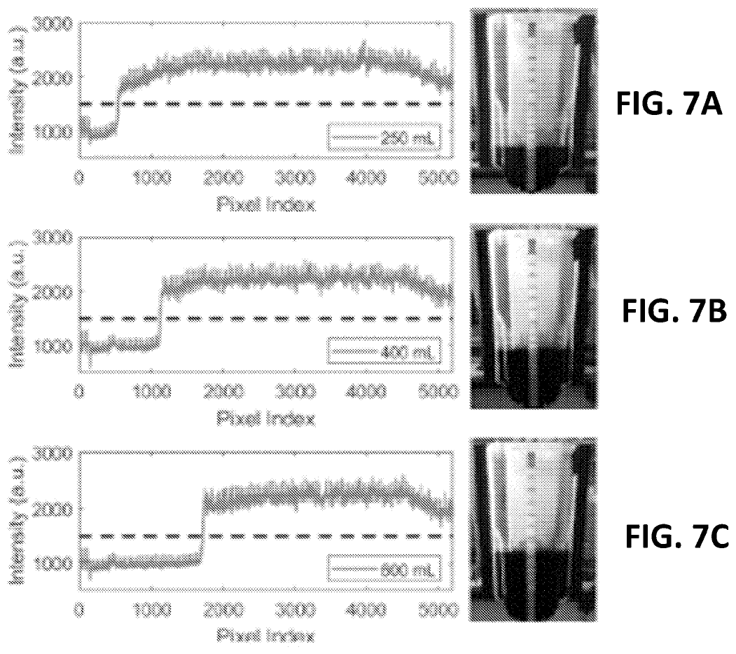

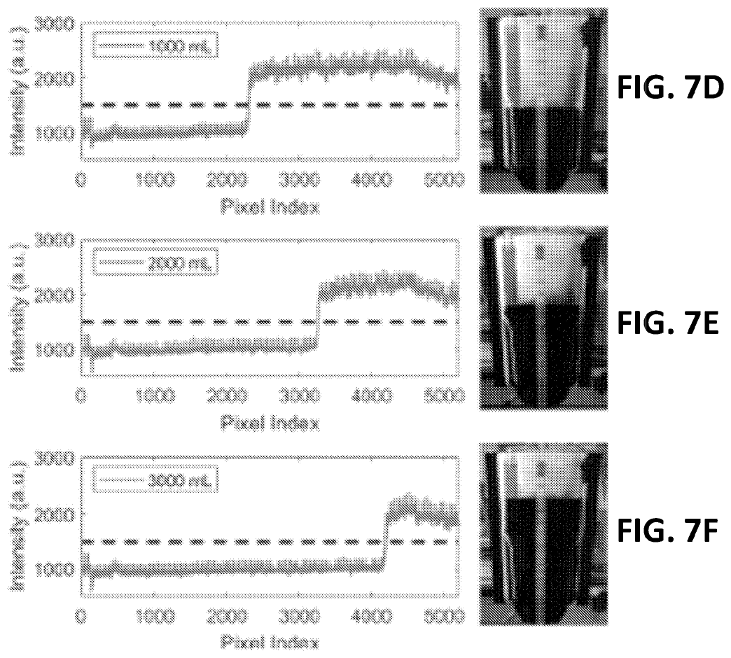

According to still other inventive embodiments, the sensor is calibrated using an opaque test liquid in dark red color, which has similar optical properties with blood. This test liquid is prepared by adding food coloring agents of red, black, and crimson colors (mixed at a ratio of 2:1:2) into water. The test liquid is loaded into the reservoir by a pump to reach a specific volume determined by the volumetric marks on the original reservoir, and the response of the CMOS sensor array is analyzed to find the count of blocked pixels. Each set of volume and count of blocked pixels give one data point in the calibration curve. By changing the volume over the entire operable range (250 ml-4,000 mL), multiple data points are collected to establish a calibration curve. FIGS. 7A-7F show example graphs of the CMOS sensor array response at different volumes. As can be seen from the graphs, the clear pixels above the blood level give much higher light intensity signal than the blocked pixels below the blood level. The transition point on the intensity graph corresponds to the blood/air interface 11 at that given volume. As the volume increases, the transition point clearly shifts towards higher pixel index which indicates the increasing count of blocked pixels. An optimal threshold of 1,500 is used to differentiate the clear and blocked pixels, and the count of blocked pixels are automatically calculated by the MCU.

To test the blood volume sensor's functions, the venous reservoir equipped with the optical blood volume sensor is used in a simulated clinical CPB procedure carried out at the Cardiovascular Perfusion SIM LAB at Comprehensive Care Services, Inc. The experiment uses a heart-lung machine (Livanova S5), a venous reservoir (Terumo CAPIOX NX19) equipped with the inventive blood volume sensor and Terumo CAPIOX FX25 Advance Oxygenator, a surgical table, and a manikin (patient simulator). The oxygenator is installed onto the reservoir which is mounted onto the post of the heart-lung machine. A traditional bypass tubing circuit is used to connect the reservoir, the oxygenator, the chest of the manikin, a centrifugal pump (Livanova REVOLUTION Blood Pump), and blood bags, into a extracorporeal circuit. Bovine blood in heparin (purchased from HemoStat Laboratories) is first loaded into the reservoir through the port on top of the arterial filter and is circulated in the extracorporeal circuit driven by the centrifugal pump. The goal for this simulated CPB procedure is to investigate how the blood volume sensor works in a clinical environment over a long period. To add blood into the reservoir or to subtract blood from the reservoir, tubings connected to the blood bags are selectively opened or closed to pump blood downward into the reservoir (to increase blood volume) or to pump blood upward into the blood bag (to decrease blood volume in the reservoir). The flow rate of blood is controlled by the centrifugal pump. Other components commonly used in clinical CPB procedures such as heat exchangers and oxygen supplies are not used in this simulation.

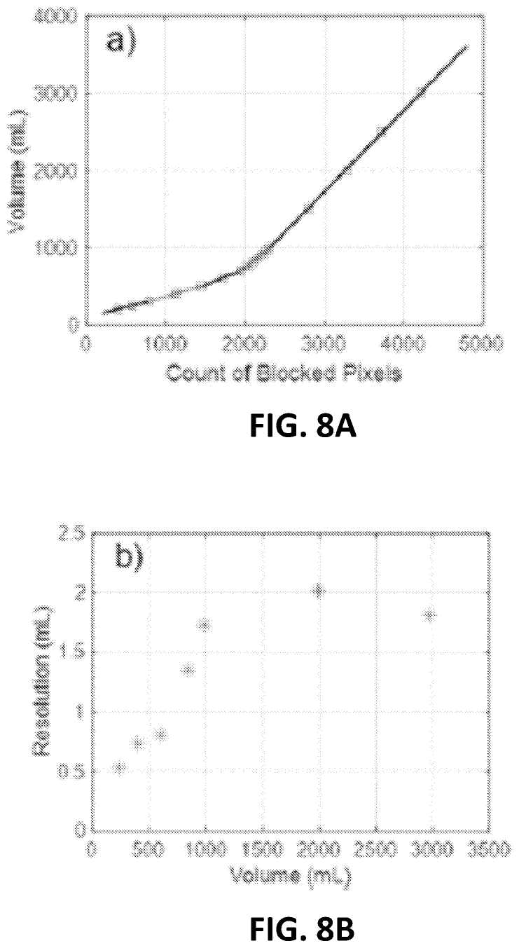

One key performance characteristic of the inventive blood volume sensor is the sensor's resolution, which is the smallest discernible volume change. Generally speaking, a sensor's resolution is mainly determined by its sensitivity and the signal-to-noise ratio. The present sensor's response is quite nonlinear and the sensitivity changes for different volume ranges, as can be seen from the five segments in FIG. 8A. Therefore, the resolution should also depend on the volume range. The resolution of the inventive sensor has been experimentally studied after the calibration procedure is completed. For reading one fixed volume, the sensor's output is recorded over a period of 30 seconds to statistically analyze the fluctuations in the measurement results. The sensor's resolution ΔV is calculated as ΔV=2σ, wherein σ is the standard deviation calculated from the collected volume readings. The resolution is plotted vs. volume in FIG. 8B. For a small volume below 700 mL the sensor's resolution is better than 1 mL. For bigger volumes (>700 mL), the sensor's resolution will decline slightly but can still resolve the volume at about 2 mL.

The non-uniform resolution can be explained from the functional mechanism of the inventive blood volume sensor. The inventive blood volume sensor optically scans the blood level and converts it into a volume reading. It can resolve the blood level with similar resolutions for different volumes. However, after converting the blood level into the volume, the resolution for larger volume is not as good as smaller volume simply because the reservoir has a larger cross-sectional area for larger volume. Compared with existing approaches of manual visual reading on the volumetric marks, the inventive blood volume sensor has improved the resolution by more than 50 times. Such a high resolution can be attributed to the small pitch size (50 μm) of the CMOS sensor pixel array. The resolution (for volume >700 mL) estimated based on the pitch size and the cross-sectional area of the reservoir is about 1 mL, which is comparable with our experimental result.

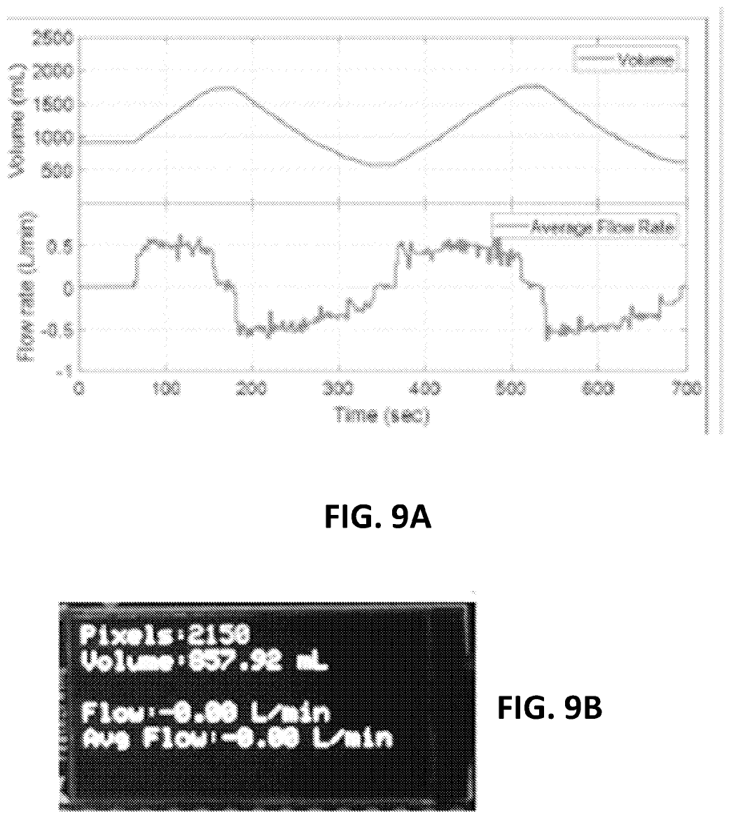

The inventive blood volume sensor has been comprehensively tested in the simulated CPB procedure. FIG. 9A shows one experimental result from the simulated CPB procedure, recorded over a period of 700 seconds. Both blood volume and average flow rate are instantly shown on the digital display which can immediately provide this crucial information of venous blood volume to the perfusionist. The information on the digital display in FIG. 9B corresponds to the moment at the beginning of this data collection period, where the blood volume remains constant, showing a 858 mL blood volume and zero flow rate. When the bovine blood began to be added into or subtracted from the reservoir, the blood volume sensor responds to the changing volume promptly. Both the blood volume and the average flow rate have been automatically tracked over time, with 4 volume readings performed in every second and one average flow rate reading in every 3 seconds. The sensor's volume readings match very closely with the volumetric marks on the reservoir with better than 97% accuracy. Similar experimental procedures have been repeated in numerous cycles and the results stayed consistent over at least three hours. A wide range of volumes has also been tested and the sensor's function can cover the entire operable range of the blood volume 250 mL-4,000 mL.

The average flow rate indicates how quickly the venous blood volume changes over time and can provide important guidance for perfusionists to make decisions during the operation. In clinical CPB procedures, this flow rate is mainly controlled through the centrifugal pump. In some procedures, the value of flow rate may be estimated by installing two separate flow rate sensors onto the tubings connected to two main ports of the reservoir, however, such an approach is generally not accurate because it cannot capture contributions from other ports of the reservoir, such as drug injection and blood transfusion into the reservoir. In comparison, using the inventive blood volume sensor, this flow rate can be reliably measured and tracked during the entire operation.

In some inventive embodiments of the inventive blood volume sensor disclosed herein offer a powerful tool for improving clinical CPB procedures. In existing clinical CPB procedures, there are already numerous data collected in real time from multiple sensors connected with the heart-lung machine, including blood pressure, oxygen saturation, temperature, critical blood level alert, bubbles detection, and blood flow rate (through certain major tubings). However, the venous blood volume has been a missing piece of information so far. With the inventive blood volume sensor installed, the venous blood volume in the reservoir becomes a new quantity that can be promptly monitored during a CPB procedure. First of all, it can significantly improve a perfusionist's efficiency and accuracy when dealing with management of patients' blood volume. The capability of automatic recording of the blood volume over a long period can be a very beneficial feature for long-term life support. For example, an extracorporeal membrane oxygenation (ECMO) support for COVID-19 patients in critical conditions may have a median duration of 18 days. The inventive blood volume sensor can effortlessly record and track the history of venous blood volume during such a long procedure.

Furthermore, the real-time information on blood volume and average flow rate measured by certain inventive embodiments of the inventive blood volume sensor open up new applications for clinical decision support engines based on machine learning platforms. One contemplated application is the prevention of CPB-related injuries. It has been widely known that injuries may occur during or after CPB procedures, such as brain injuries, lung injuries, and kidney injuries. The fundamental reasons for such injuries still require further research. For example, brain injuries are believed to be caused by a reduced cerebral blood flow (CBF) during a CPB procedure. With the inventive blood volume sensor, the venous blood volume in the reservoir and the net flow rate are be introduced into the equations for modelling the CPB procedures to quantitatively investigate the causes of injuries. Together with all other measurable quantities, the physiology of the patient under CPB are precisely and quantitatively modelled, in order to study, and predict physiological changes during CPB procedures.

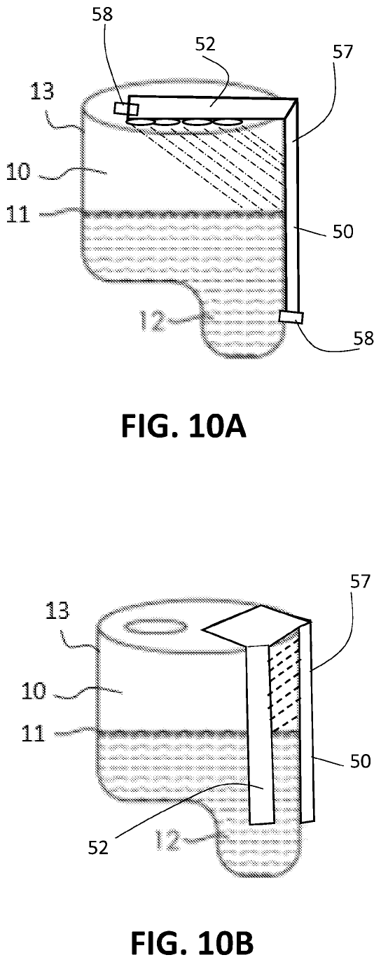

According to certain inventive embodiments as shown in FIGS. 10A and 10B, the volume sensor 2 is a variation on the above described embodiments of an optical blood volume sensor. That is, as shown in FIG. 10A, the CIS sensor 50 and the light source 52 are provided on an L-shaped bracket 57 with the light source 52 positioned along the top of the container 13 so that the lights cast light into the reservoir 10 and so that the CIS 50 is positioned along the height of the container 13. Notably, clamps 58 for mounting the bracket 57 on the container 13 are provided. The light source 52 casts light into the reservoir 10 toward the CIS 50. As described above, the light is not able to pass through the blood within the reservoir 10 thereby blocking the CIS 50 from perceiving the light below the level of the blood within the reservoir 10. Thus, the volume of the blood 12 in the reservoir 10 is calculated. As shown in FIG. 10B, the CIS sensor 50 and the light source 52 are provided on a bracket 57 with a portion that attaches to the top of the container 13 and with portions that extend therefrom along the height of the container 13 and on which the light source 52 and the CIS 50 are positioned, respectively so that the lights of the light source 50 cast light into the reservoir 10 toward the CIS. As described above, the light is not able to pass through the blood within the reservoir 10 thereby blocking the CIS 50 from perceiving the light below the level of the blood within the reservoir 10. Thus, the volume of the blood 12 in the reservoir 10 is calculated.

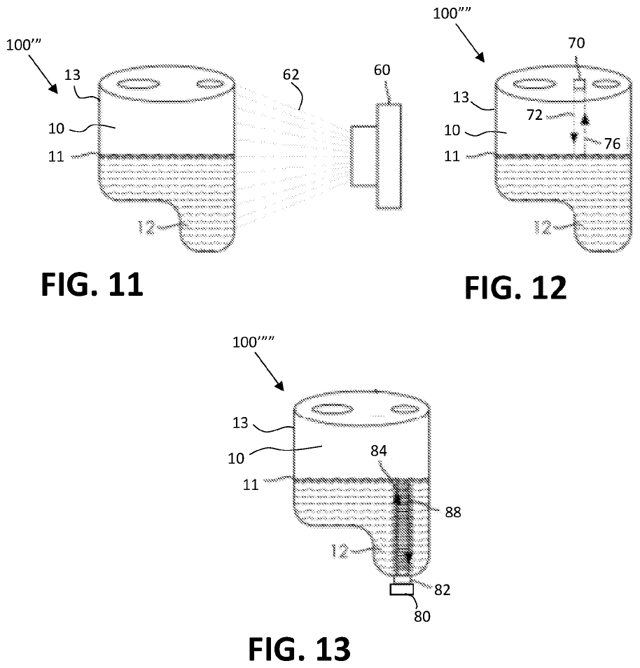

According to an inventive embodiment as shown in FIG. 11, the volume sensor 2 of an inventive blood volume sensor system 100, as described above, is a camera device 60. In such camera based blood volume sensor systems 100′″, the camera sensor 60 is positioned such that it has a view of the blood reservoir 10. The camera sensor 60 then directly images the reservoir 10 and analyzes the image of the blood/air interface 11 formed within the reservoir 10 at the level of the blood 12. This processes information is used to calculate the level of the blood 12 within the reservoir 10 and the volume of the blood 12 within the reservoir 10. According to other inventive embodiments, the camera sensor 60 is standard camera that images the reservoir 10. According to further inventive embodiments, the camera based blood volume sensor systems 100′″ includes a laser beam 62 emitted from a laser source and a line scan camera 60. According to these inventive embodiments, the laser source emits a laser beam 62 toward the reservoir 10. The laser beam 62 is reflected from blood 12 in the reservoir 10 back to the camera, while the laser beam 62 passes through the reservoir 10 where no blood is present. Accordingly, the laser beam 62 that is reflected back to and detected by the camera 60 provides data regarding the level of blood 12 within the reservoir 10. This data is then used to calculate the level and volume of blood 12 within the reservoir 10. The camera sensor 60 communicates this data to a computer 4 or other suitable digital computing device, as described above.

According to an inventive embodiment as shown in FIG. 12, the volume sensor 2 of an inventive blood volume sensor system 100, as described above, is a time of flight (TOF) laser sensor 70. In such TOF laser based blood volume sensor systems 100″″, the TOF laser sensor 70 is positioned at the top of the reservoir 10 such that it has a view of any blood 12 contained therein. According to other inventive embodiments, the TOF laser sensor 70, which a sensor configured to measure distance, is mounted on the exterior of the reservoir 10 container, which according to other inventive embodiments is transparent. The TOF laser sensor 70 sends a laser pulse 72 towards the blood 12 contained in the reservoir 10 below. The laser pulse 72 is reflected by the blood 12 at the blood/air interface 11 back to the TOF laser sensor 70. The reflected laser pulse 76 is then detected by the TOF laser sensor 70. The distance from the sensor 70 to the blood/air interface 11 is measured based on the time of flight in the round trip of the laser pulse 72, 76 and the change of beam intensity. From the measured time of flight distance, the blood level and blood volume are calculated. The TOF laser sensor 70 communicates this data to a computer 4 or other suitable digital computing device, as described above.

According to an inventive embodiment as shown in FIG. 13, the volume sensor 2 of an inventive blood volume sensor system 100, as described above, is an ultrasound time of flight (TOF) sensor 80. In such ultrasound TOF based blood volume sensor systems 100′″″, the ultrasound TOF sensor 80, also known as an ultrasound transducer, is positioned at the bottom of the reservoir 10 such that it is configured to emit ultrasonic waves into any blood 12 contained therein. According, to other inventive embodiments, the ultrasound TOF sensor 80 is mounted on the exterior of the bottom of the reservoir 10, which according to some inventive embodiments allows efficient coupling of ultrasound waves into the blood. An acoustic impedance matching layer 82 is provided between the ultrasound TOF sensor 80 and the reservoir 10 to allow all ultrasound waves to be emitted into the blood 12 contained in the reservoir 10. The ultrasound TOF sensor 80 sends a pulse of ultrasound wave 84 into the blood 12 contained in the reservoir 10. The ultrasound wave 84 is then reflected back to the sensor 80 when the pulse reaches the blood/air interface 11. The reflected ultrasound wave pulse 88 is then detected by the sensor 80. The distance from the sensor 80 to the blood/air interface 11 is measured based on the time of flight in the round trip of the ultrasound wave 84, 88. From the measured distance, the level and volume of the blood 12 in the reservoir 10 is then calculated. The ultrasound TOF sensor 80 communicates this data to a computer 4 or other suitable digital computing device, as described above.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention but is not meant to be a limitation upon the practice thereof. The following claims, including all equivalents thereof, are intended to define the scope of the invention.

1. A blood volume sensor system comprising:
a container defining a blood volume reservoir;
a volume sensor configured to monitor the blood volume reservoir and output data corresponding to a volume of blood contained in the blood volume reservoir; and
a digital computing device in communication with the volume sensor and configured to receive the output data regarding the volume of blood contained in the blood volume reservoir.

2. The blood volume sensor system of claim 1 wherein the volume sensor is a gravimetric blood volume sensor.

3. The blood volume sensor system of claim 2 wherein the gravimetric blood volume sensor includes a load cell configured to measure a weight of the blood contained in the blood volume reservoir.

4. The blood volume sensor system of claim 3 wherein the load cell is coupled to the container defining the blood volume reservoir or a support structure.

5. (canceled)

6. The blood volume sensor system of claim 3 wherein the load cell communicates with a microcontroller unit.

7. The blood volume sensor system of claim 1 wherein the volume sensor is an optical sensor.

8. The blood volume sensor system of claim 7 wherein the optical sensor is a contact imaging sensor.

9. The blood volume sensor system of claim 8 wherein contact imaging sensor includes an array of photodiodes configured to detect light emitted by a light source.

10. The blood volume sensor system of claim 9 wherein the light source is positioned along a height of the reservoir or along a top or the reservoir and is configured to shine light through the reservoir.

11. The blood volume sensor system of claim 9 wherein the array of photodiodes is positioned along a height of the reservoir.

12. The blood volume sensor system of claim 9 wherein the array of photodiodes includes a linear array of more than 5,000 CMOS sensor pixels.

13. (canceled)

14. The blood volume sensor system of claim 7 wherein the optical sensor communicates with a microcontroller unit.

15. The blood volume sensor system of claim 9 wherein the contact imaging sensor additionally includes at least one layer of neutral density filter film positioned on the contact imaging sensor.

16. The blood volume sensor system of claim 1 wherein the volume sensor is a camera sensor.

17. (canceled)

18. The blood volume sensor system of claim 1 wherein the volume sensor is a time of flight laser sensor.

19. The blood volume sensor system of claim 1 wherein the volume sensor is an ultrasound time of flight sensor.

20. The blood volume sensor system of claim 1 further comprising an oxygenator attached to the blood volume reservoir.

21. The blood volume sensor system of claim 1 wherein the container is transparent.

22. The blood volume sensor system of claim 1 wherein the blood volume sensor is incorporated into a surgical pack of a heart lung machine.

23. The blood volume sensor system of claim 1 wherein the volume sensor monitors the blood volume reservoir in real time.

24. (canceled)

25. (canceled)

26. (canceled)
